(19) United States
(12) Patent Application Publication
(10) Pub. No.: US 2019/0151640 A1
(43) Pub. Date: May 23, 2019

(54) INTERACTIVE WEARABLE AND E-TATTOO COMBINATIONS

(71) Applicant: Boston Scientific Scimed, Inc., Maple Grove, MN (US)

(72) Inventors: Jan Weber, Maastricht (NL); Keith R. Maile, New Brighton, MN (US); Deepa Mahajan, North Oaks, MN (US); Jeffrey E. Stahmann, Ramsey, MN (US); James M. Peck, Maple Grove, MN (US)

(21) Appl. No.: 16/196,934

(22) Filed: Nov. 20, 2018

Related U.S. Application Data
(60) Provisional application No. 62/589,131, filed on Nov. 21, 2017.

(51) Int. Cl. A61M 37/00; H04M 1/725; H04M 1/05

(52) U.S. Cl. CPC A61M 37/0076; H04M 1/05; H04M 1/7253

(57) ABSTRACT

A system for monitoring one or more respiratory conditions is disclosed. The system comprises a medical device and an e-tattoo configured to attach to the subject's skin. The medical device is configured to monitor at least one respiratory parameter of a subject and transmit a signal in response to monitoring a trigger event in the at least one respiratory parameter. The e-tattoo is configured to: receive the signal from the medical device; sense at least one environmental parameter in response to receiving the signal; and transmit the at least one environmental parameter.

### CROSS-REFERENCE TO RELATED APPLICATION

This application claims priority to Provisional Application No. 62/589,131, filed Nov. 21, 2017, which is herein incorporated by reference in its entirety.

### TECHNICAL FIELD

The present disclosure relates to devices and methods for monitoring one or more physiological parameters of a subject. More specifically, the disclosure relates to devices, systems, and methods for monitoring respiration using a medical device and an e-tattoo.

### BACKGROUND

Some respiratory conditions such as Chronic Obstructive Pulmonary Disease (COPD) may be long-term, progressive lung conditions characterized by airway inflammation that impedes breathing. The airway inflammation is not fully reversible and environmental conditions can exasperate airway inflammation worsening the condition. Managing such conditions may include physiological monitoring.

### SUMMARY

Embodiments included herein facilitate monitoring respiratory conditions (e.g., COPD) and environmental conditions that may worsen respiratory conditions. Monitoring respiratory and environmental conditions that may worsen respiratory conditions may result in better management of the condition.

In an Example 1, a system for monitoring one or more respiratory conditions comprises: a medical device configured to: monitor at least one respiratory parameter of a subject; and transmit a signal in response to monitoring a trigger event in the at least one respiratory parameter; and an e-tattoo configured to attach to the subject's skin, the e-tattoo configured to: receive the signal from the medical device; sense at least one environmental parameter in response to receiving the signal; and transmit the at least one environmental parameter.

In an Example 2, the system of Example 1, wherein the medical device is configured to receive the at least one transmitted environmental parameter.

In an Example 3, the system of Example 2, wherein the medical device is configured to modify a therapy in response to receiving the at least one transmitted environmental parameter.

In an Example 4, the system of either of Examples 2 or 3, wherein the medical device is further configured to: determine the at least one environmental parameter is an adverse environmental parameter; and provide, to the subject, an indication the at least one environmental parameter is an adverse environmental parameter.

In an Example 5, the system of any of Examples 1-4, wherein the at least one environmental parameter is at least one of: a particulate and a volatile organic compound.

In an Example 6, the system of any of Examples 1-5, wherein the trigger event is at least one of: a change in a respiratory sound and a change in respiratory rate.

In an Example 7, the system of any of Examples 1-6, wherein the e-tattoo is further configured to: sense a baseline measurement of a respiratory parameter, wherein the baseline measurement is sensed prior to the medical device modifying a therapy; sense a non-baseline measurement of the respiratory parameter, wherein the non-baseline measurement is sensed after the medical device modifies a therapy; and transmit the baseline measurement and the non-baseline measurement to a device.

In an Example 8, the system of Example 7, wherein the device is configured to: compare the baseline measurement and the non-baseline measurement; and modify the therapy for the subject in response to the comparison.

In an Example 9, the system of either of Examples 7 or 8, further comprising a display device communicatively coupled to at least one of: the medical device and the e-tattoo, the display device configured to present a representation of at least one of: the at least one environmental parameter, the baseline measurement, and the non-baseline measurement.

In an Example 10, a method for monitoring one or more respiratory conditions comprises: receiving, by an e-tattoo attachable to a subject's skin, a signal transmitted from a medical device, wherein the signal is transmitted in response to detecting a trigger event in at least one respiratory parameter of a subject; sensing at least one environmental parameter in response to receiving the signal; and transmitting the at least one environmental parameter.

In an Example 11, the method of Example 10, further comprising: sensing a baseline measurement of a respiratory parameter, wherein the baseline measurement is sensed prior to the medical device modifying a therapy; sensing a non-baseline measurement of the respiratory parameter, wherein the non-baseline measurement is sensed after the medical device delivers the therapy; and transmitting the baseline measurement and the non-baseline measurement to a device.

In an Example 12, the method of Example 11, further comprising: comparing the baseline measurement and the non-baseline measurement; and modifying the therapy for the subject in response to the comparison.

In an Example 13, the method of either of Examples 11 or 12, further comprising displaying a representation of at least one of: the at least one environmental parameter, the baseline measurement, and the non-baseline measurement.

In an Example 14, the method of any of Examples 10-13, wherein the trigger event is at least one of: a change in a respiratory sound and a change in respiratory rate.

In an Example 15, the method of any of Examples 10-14, wherein the at least one environmental parameter is at least one of: a particulate and a volatile organic compound.

In an Example 16, a system for monitoring one or more respiratory conditions comprises: a medical device configured to: monitor at least one respiratory parameter of a subject of a subject; and transmit a signal in response to monitoring a trigger event in the at least one respiratory parameter; and an e-tattoo configured to attach to the subject's skin, the e-tattoo configured to: receive the signal from the medical device; sense at least one environmental parameter in response to receiving the signal; and transmit the at least one environmental parameter.

In an Example 17, the system of Example 16, wherein the medical device is configured to receive the at least one transmitted environmental parameter.

In an Example 18, the system of Example 17, wherein the medical device is configured to modify a therapy in response to receiving the at least one transmitted environmental parameter.

In an Example 19, the system of Example 17, wherein the medical device is further configured to: determine the at least one environmental parameter is an adverse environmental parameter; and provide, to a subject, an indication the at least one environmental parameter is an adverse environmental parameter.

In an Example 20, the system of Example 16, wherein the at least one environmental parameter is at least one of: a particulate and a volatile organic compound.

In an Example 21, the system of Example 16, wherein the trigger event is at least one of: a change in a respiratory sound and a change in respiratory rate.

In an Example 22, the system of Example 16, wherein the e-tattoo is further configured to: sense a baseline measurement of a respiratory parameter, wherein the baseline measurement is sensed prior to the medical device modifying a therapy; sense a non-baseline measurement of the respiratory parameter, wherein the non-baseline measurement is sensed after the medical device modifies a therapy; and transmit the baseline measurement and the non-baseline measurement to a device.

In an Example 23, the system of Example 22, wherein the device is configured to: compare the baseline measurement and the non-baseline measurement; and modify the therapy for the subject in response to the comparison.

In an Example 24, the system of Example 22, further comprising a display device communicatively coupled to at least one of: the medical device and the e-tattoo, the display device configured to present a representation of at least one of: the at least one environmental parameter, the baseline measurement, and the non-baseline measurement.

In an Example 25, a method for monitoring one or more respiratory conditions comprises: receiving, by an e-tattoo configured to attach to a subject's skin, a signal transmitted from a medical device, wherein the signal is transmitted in response to detecting a trigger event in at least one respiratory parameter of a subject; sensing at least one environmental parameter in response to receiving the signal; and transmitting the at least one environmental parameter.

In an Example 26, he method of Example 25, further comprising: sensing a baseline measurement of a respiratory parameter, wherein the baseline measurement is sensed prior to the medical device modifying a therapy; sensing a non-baseline measurement of the respiratory parameter, wherein the non-baseline measurement is sensed after the medical device delivers the therapy; and transmitting the baseline measurement and the non-baseline measurement of a respiratory parameter to a device.

In an Example 27, the method of Example 26, further comprising: comparing the baseline measurement and the non-baseline measurement; and modifying the therapy for the subject in response to the comparison.

In an Example 28, the method of Example 26, further comprising displaying a representation of at least one of: the at least one environmental parameter, the baseline measurement, and the non-baseline measurement.

In an Example 29, the method of Example 25, wherein the trigger event is at least one of: a change in a respiratory sound and a change in respiratory rate.

In an Example 30, the method of Example 25, wherein the at least one environmental parameter is at least one of: a particulate and a volatile organic compound.

In an Example 31, an e-tattoo comprises: an adhering mechanism for attaching the e-tattoo to a subject's skin; and a processing device configured to: receive an signal from a medical device; sense at least one environmental parameter in response to receiving the signal; and transmit the at least one environmental parameter.

In an Example 32, the e-tattoo of Example 31, wherein the processing device is further configured to: sense a baseline measurement of a respiratory parameter, wherein the baseline measurement is sensed prior to the medical device modifying a therapy; sense a non-baseline measurement of the respiratory parameter, wherein the non-baseline measurement is sensed after the medical device modifies a therapy; and transmit the baseline measurement and the non-baseline measurement to a device.

In an Example 33, the e-tattoo of Example 32, wherein the processing device is further configured to: compare the baseline measurement and the non-baseline measurement and transmit the comparison to the device.

In an Example 34, the e-tattoo of Example 31, wherein the processing device is further configured to: determine the at least one environmental parameter is an adverse environmental parameter; and transmit a signal to the device indicating the determined adverse environmental parameter.

In an Example 35, the e-tattoo of Example 31, wherein the at least one environmental parameter is at least one of: a particulate and a volatile organic compound.

While multiple embodiments are disclosed, still other embodiments of the presently disclosed subject matter will become apparent to those skilled in the art from the following detailed description, which shows and describes illustrative embodiments of the disclosed subject matter. Accordingly, the drawings and detailed description are to be regarded as illustrative in nature and not restrictive.

### BRIEF DESCRIPTION OF THE DRAWINGS

FIG. 1 is a schematic diagram of an illustrative physiological monitoring system, in accordance with embodiments of the subject matter disclosed herein.

FIG. 2 is a block diagram depicting an illustrative operating environment, in accordance with embodiments of the subject matter disclosed herein.

FIG. 3 is a flow diagram depicting an illustrative method for monitoring a subject and/or a subject's environment, in accordance with embodiments disclosed herein.

FIG. 4 is a flow diagram depicting another illustrative method for monitoring a subject and/or a subject's environment, in accordance with embodiments disclosed herein.

While the disclosed subject matter is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

As used herein in association with values (e.g., terms of magnitude, measurement, and/or other degrees of qualitative and/or quantitative observations that are used herein with respect to characteristics (e.g., dimensions, measurements, attributes, components, etc.) and/or ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a value, configuration, orientation, and/or other characteristic that is equal to (or the same as) the stated value, configuration, orientation, and/or other characteristic or equal to (or the same as) a value, configuration, orientation, and/or other characteristic that is reasonably close to the stated value, configuration, orientation, and/or other characteristic, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

Although the term “block” may be used herein to connote different elements illustratively employed, the term should not be interpreted as implying any requirement of, or particular order among or between, various blocks disclosed herein. Similarly, although illustrative methods may be represented by one or more drawings (e.g., flow diagrams, communication flows, etc.), the drawings should not be interpreted as implying any requirement of, or particular order among or between, various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As used herein, the term “based on” is not meant to be restrictive, but rather indicates that a determination, identification, prediction, calculation, and/or the like, is performed by using, at least, the term following “based on” as an input. For example, predicting an outcome based on a particular piece of information may additionally, or alternatively, base the same determination on another piece of information.

### DETAILED DESCRIPTION

Embodiments disclosed herein provide systems and methods for monitoring one or more respiratory conditions. In embodiments, the systems and methods may use two or more devices to monitor the respiratory conditions. Each of the two or more devices may have advantages and/or disadvantages as compared to the other device. For example, in an embodiment, a first device may be an e-tattoo while a second device may be a medical device and/or an implantable medical device. The flexible structure of the e-tattoo may allow the e-tattoo to be placed in positions on a subject where the second device may not be able to be placed and/or where it may be uncomfortable for the subject. As such, the e-tattoo may be able to sense parameters that the second device may not be able to sense. Additionally or alternatively, while the e-tattoo may be able to sense parameters the second device may not be able to sense, in embodiments, the other device may have longer battery life, better processing power, be able to provide more or better therapies, rely less on patient compliance, sense parameters the e-tattoo cannot sense and/or have other functionality that the e-tattoo may not be able to accommodate due to its flexible nature or otherwise. Additionally or alternatively, the second device may be implanted within a subject to determine the occurrence of one or more episodes of a respiratory condition and, in response, the e-tattoo, which may be placed on the skin of a subject, may be configured to sense environmental parameters that may be contributing to and/or worsening one or more respiratory conditions. These are only examples of some of the advantages of the embodiments disclosed herein, though, and should not be considered limiting.

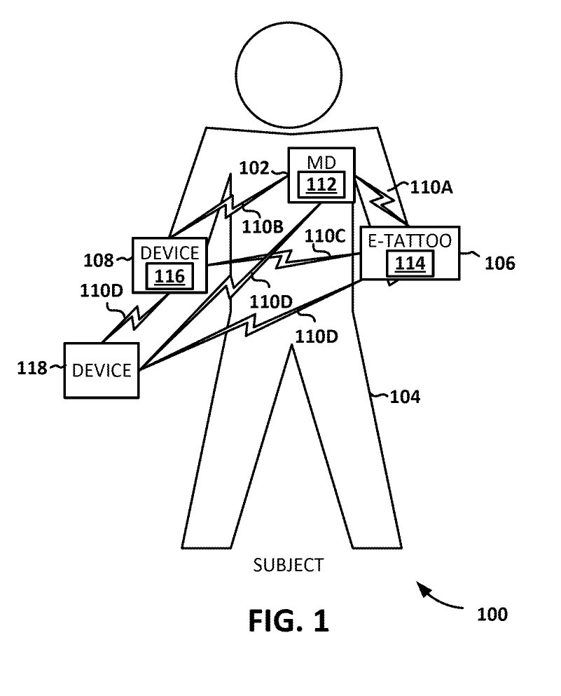

FIG. 1 shows an illustrative physiological monitoring system 100, in accordance with embodiments of the disclosure. As shown in FIG. 1, the system 100 includes a medical device (MD) 102 configured to be positioned adjacent the body of a subject 104, disposed on the body of the subject 104, and/or implanted within the body of a subject 104. The system 100 also includes an e-tattoo 106. The e-tattoo 106 may be positioned adjacent the body of a subject 104, disposed on the body of the subject 104, and/or implanted within the body of a subject 104. In embodiments, the medical system also includes another device 108. The device 108 may be positioned adjacent the body of a subject 104, disposed on the body of the subject 104, and/or implanted within the body of a subject 104. The subject 104 may be a human, a dog, a pig, and/or any other animal having physiological parameters that can be recorded. For example, in embodiments, the subject 104 may be a human patient.

In embodiments, the MD 102 and the e-tattoo 106 may be communicatively coupled via a communication link 110A. In embodiments, the MD 102 and/or the e-tattoo 106 may also be coupled to a device 108 via communication links 110B, 110C, respectively. The communication links 110A, 110B, 110C may be the same type of communication link or different types of communication links. In embodiments, the communication links 110A, 110B, 110C may be, or include, a wired link (e.g., a link accomplished via a physical connection) and/or a wireless communication link such as, for example, a short-range radio link, such as Bluetooth, Bluetooth Low Energy, IEEE 802.11, near-field communication (NFC), WiFi, a proprietary wireless protocol, and/or the like. The term “communication link” may refer to an ability to communicate some type of information in at least one direction between at least two devices, and should not be understood to be limited to a direct, persistent, or otherwise limited communication channel. That is, according to embodiments, the communication links 110A, 110B, 110C may be a persistent communication link, an intermittent communication link, an ad-hoc communication link, and/or the like. The communication links 110A, 110B, 110C may refer to direct communications between the MD 102, the e-tattoo 106 and/or the device 108, and/or indirect communications that travel between the MD 102, the e-tattoo 106 and/or the device 108 via at least one other device (e.g., a repeater, router, hub, and/or the like). The communication links 110A, 110B, 110C may facilitate uni-directional and/or bi-directional communication between the MD 102, the e-tattoo 106 and/or the device 108. Data and/or control signals may be transmitted between the MD 102, the e-tattoo 106, and the device 108 to coordinate the functions of the MD 102, the e-tattoo 106, and/or the device 108. In embodiments, subject data may be downloaded from one or more of the MD 102, the e-tattoo 106, and/or the device 108 periodically or on command. The clinician and/or the subject 104 may communicate with the MD 102, the e-tattoo 106, and/or the device 108, for example, to acquire subject data or to initiate, terminate and/or modify recording and/or therapy. In embodiments, the communication links 110A, 110B, 110C may facilitate encryption and/or other methods to increase data transmission safety.

In embodiments, the MD 102, the e-tattoo 106 and/or the device 108 may provide one or more of the following functions with respect to a subject: sensing, data storage, data analysis and/or presentation, and therapy. For example, in embodiments, the MD 102, the e-tattoo 106, and/or the device 108 may be used to measure any number of a variety of physiological, device, subjective, and/or environmental parameters associated with the subject 104, using electrical, mechanical, optical, and/or chemical means. The MD 102, the e-tattoo 106, and/or the device 108 may be configured to automatically gather data, gather data upon request (e.g., input provided by the subject, a clinician, another device, and/or the like), gather data in response to an event, and/or any number of various combinations and/or modifications thereof. The MD 102, the e-tattoo 106, and/or the device 108 may be configured to store data related to the physiological, device, environmental, and/or subjective parameters and/or transmit the data to any number of other devices in the system 100. In embodiments, the MD 102, the e-tattoo 106, and/or the device 108 may be configured to analyze data and/or act upon the analyzed data. For example, the MD 102, the e-tattoo 106, and/or the device 108 may be configured to modify therapy, perform additional monitoring, store pre-analyzed or post-analyzed data, and/or provide alarm indications based on the analysis of the data.

In embodiments, the MD 102, the e-tattoo 106, and/or the device 108 may be configured to provide therapy. Therapy may be provided autonomously and/or upon request (e.g., an input by the subject 104, a clinician, another device or process, and/or the like). The MD 102, the e-tattoo 106, and/or the device 108 may be programmable in that various characteristics of their sensing, therapy (e.g., duration and interval), and/or communication may be altered by communication between the devices 102, 106, 108 and/or other components of the system 100. For example, in embodiments, a first e-tattoo 106 may be configured to communicate with a second e-tattoo 106 to trigger the second e-tattoo to perform an action (e.g., a sensing action, a therapy action, etc.). In this manner, for example, timing of various activities performed by e-tattoos may be configured and maintained based on a communication scheme involving a number of the devices.

According to embodiments, the MD 102 may include any type of medical device (e.g., a wearable medical device (WMD), an implantable medical device (IMD), etc.) that senses one or more physiological signals of the subject 104, administers one or more therapies, and/or the like, and may include any number of different components of a medical device. For example, the MD 102 may include a control device, a monitoring device, a respiratory device, a pacemaker, an implantable cardioverter defibrillator (ICD), a cardiac resynchronization therapy (CRT) device, a neurostimulation device, a drug delivery device, a muscular stimulation device, an optimal or audio stimulation device, and/or the like, and may be a medical device known in the art or later developed, for sensing physiological signals, providing therapy and/or diagnostic data about the subject 104 and/or the MD 102. In various embodiments, the MD 102 may include a drug delivery functionality (e.g., an inhaler functionality, a nebulizer functionality and/or the like), ventilating functionality, defibrillation, an air filtration functionality, a smoking cessation functionality, an oxygen delivery functionality, a volatile compound release functionality, and/or pacing/CRT capabilities (e.g., a CRT-D device). In embodiments, the MD 102 may be implanted subcutaneously within an implantation location or pocket in the patient's chest or abdomen and may be configured to monitor (e.g., sense and/or record) physiological parameters associated with one or more body systems of the subject 104 (e.g., the respiratory system, the nervous system, and/or the circulatory system). In embodiments, the MD 102 may be an implantable respiratory monitor, an implantable cardiac monitor (ICM) (e.g., an implantable diagnostic monitor (IDM), an implantable loop recorder (ILR), etc.) configured to record physiological parameters such as, for example, one or more respiratory signals, cardiac electrical signals, spirometry, oximetry, arterial blood gas measurements, heart sounds, heart rate, blood pressure measurements, oxygen saturations, and/or the like.

According to embodiments, the e-tattoo 106 may include any number of different types of devices configured to be placed on, coupled to, embedded in, and/or otherwise interfaced with a subject's body (e.g., skin). In embodiments, the e-tattoo 106 is a relatively flat device resembling a tattoo or sticker. The e-tattoo 106 may be configured to include circuitry that facilitates sensing and/or therapy functions. In embodiments, the e-tattoo 106 may include an adhesive layer that facilitates the e-tattoo 106 being attached to the subject 104. Additionally or alternatively, the e-tattoo 106 may be attached to the subject 104 using another adhesive and/or compound not included in the e-tattoo 106. Additionally or alternatively, the e-tattoo 106 may be stamped and/or printed on the subject 104.

According to embodiments, the e-tattoo 106 includes any number of different types of sensors, circuits, processing devices, chemical and/or biochemical depots, energy sources and/or memory. For example, the e-tattoo 106 may include sensors configured to sense one or more environmental parameters to which the subject 104 is exposed and/or physiological parameters of the subject 104. The environmental parameters may include particulates, ultraviolet light, volatile organic compounds, and/or the like in the environment. The physiological parameters may include respiratory parameters (e.g., rate, depth, rhythm), motion parameters, (e.g., walking, running, falling, gait, gait rhythm), facial expressions, swelling, heart sounds, sweat, sweat composition (e.g., ammonia, pH, potassium, sodium, chloride), exhaled air composition, Electrocardiography (ECG) parameters, electroencephalogram (EEG) parameters, Electromyography (EMG) parameters, and/or the like. In embodiments, the e-tattoo 106 may include processing devices configured to process the sensed parameters, memory to store the sensed parameters, transmitters to transmit the sensed parameters, and/or receivers to receive one or more transmissions.

In embodiments, the e-tattoo 106 may be deformable so that the e-tattoo 106 is able to form to different contours of a subject 104 and/or flex and/or stretch, thereby accommodating movement of the subject 104. Comparatively, in embodiments, the MD 102 and/or the device 108 may have a more rigid structure. Due to the e-tattoo's 106 deformation ability, the e-tattoo 106 may be able to be placed on different areas of the subject 104. For example, the e-tattoo 106 may be placed on one or more of the following areas of the subject 104: abdomen, chest, back, wrist, thigh, calve, foot, ankles, arm, hands, eyelids, ears, earlobes, penis, forehead, neck, and/or the like. These placements may facilitate sensing one or more environmental and/or physiological parameters set forth above.

Additionally or alternatively, the e-tattoo 106 may allow gases and/or liquids to permeate all or certain portions of the e-tattoo 106. The gas and/or liquid flow may be bidirectional or unidirectional. The e-tattoo 106 may allow some gases and/or liquids to permeate the e-tattoo 106 while preventing flow of other gases and/or liquids.

While one e-tattoo 106 is depicted in FIG. 1, in embodiments, there may be multiple e-tattoos 106 positioned adjacent the body of a subject 104, disposed on the body of the subject 104, and/or implanted within the body of a subject 104. In embodiments, each e-tattoo 106 may be configured to perform the same function as the other e-tattoos 106 or perform different functions from the same or different locations. For example, an e-tattoo 106 may be placed on the chest of the subject 104 to record thoracic sounds and an e-tattoo 106 may be placed on the eyelid of the subject 104 to record eye movements (e.g., eyelid movements) indicative of REM sleep. Additionally or alternatively, a series of e-tattoos 106 may be placed (e.g., subsequently in time) on a subject whereby information from a first e-tattoo 106 may be used to adjust and/or modify functionality of an additional e-tattoo 106 such as, for example, an additional tattoo that was placed on the subject 104 at a later time than that of the first e-tattoo 106, that is configured to perform an action at a later time than the first e-tattoo, and/or the like.

According to embodiments, the device 108 may be a wearable device (e.g., smartwatch), a portable computing device (e.g., smartphone), and/or the like. Additionally or alternatively, the device 108 may include any type of medical device (e.g., a wearable medical device (WMD), an implantable medical device (IMD), etc.), any number of different components of a wearable device, any number of different components of an implantable system, and/or the like. For example, the device 108 may include a control device, a monitoring device, a respiratory device, a pacemaker, an implantable cardioverter defibrillator (ICD), a cardiac resynchronization therapy (CRT) device and/or the like, and may be a wearable device and/or an implantable medical device known in the art or later developed, for sensing physiological parameters of the subject 104, providing therapy and/or diagnostic data about the subject 104 and/or the device 108. In various embodiments, the device 108 may include inhaler functionality, nebulizer functionality, ventilating functionality, defibrillation, and pacing/CRT capabilities (e.g., a CRT-D device). In embodiments, the device 108 may be wearable on the subject 104 and/or implanted subcutaneously within an implantation location or pocket in subject 104 (e.g., chest or abdomen) and may be configured to monitor (e.g., sense and/or record) physiological parameters associated with subject 104 (e.g., respiratory system, and/or circulatory system). In embodiments, the device 108 may be an implantable respiratory monitor, an implantable cardiac monitor (ICM) (e.g., an implantable diagnostic monitor (IDM), an implantable loop recorder (ILR), etc.) configured to record physiological parameters such as, for example, one or more respiratory signals, cardiac electrical signals, spirometry, oximetry, arterial blood gas measurements, heart sounds, heart rate, blood pressure measurements, oxygen saturations, and/or the like.

In various embodiments, the MD 102, the e-tattoo 106, and/or the device 108 may be a device that is configured to be portable with the subject 104, e.g., by being attached to the subject 104 and/or integrated into a vest, belt, harness, sticker; placed into a pocket, a purse, or a backpack; carried in the subject's hand; and/or the like, or otherwise operatively (and/or physically) coupled to the subject 104. The MD 102, the e-tattoo 106, and/or the device 108 may be configured to monitor (e.g., sense and/or record) different physiological parameters associated with the subject 104, environmental parameters, and/or provide therapy to the subject 104. In embodiments, the MD 102, the e-tattoo 106, and/or the device 108 may provide any number of different therapy components such as, for example, an inhaler component, a nebulizer component, a defibrillation component, a drug delivery component, a neurostimulation component, a neuromodulation component, a temperature regulation component, and/or the like. In embodiments, e-tattoo 106 may include limited functionality, e.g., environmental parameter sensing activity and communication capabilities, with respiratory functionality detection, classification and/or therapy command/control being performed by a separate device such as, for example, the MD 102 and/or the device 108. In embodiments, the MD 102 may include limited functionality, e.g., detecting respiratory functionality, therapy command/control and communication capabilities, with the analysis, classification, and alert functionality of the detected data being performed by a separate device such as, for example, the device 108.

In addition, the MD 102, e-tattoo 106, and/or the device 108 may include one or more sensors 112, 114, 116, respectively, configured to detect a variety of physiological signals and/or environmental signals that may be used in connection with various diagnostic, therapeutic and/or monitoring implementations. For example, the MD 102, the e-tattoo 106, and/or the device 108 may include sensors or circuitry for detecting respiratory system signals, cardiac system signals, heart sounds, and/or signals related to patient activity. In embodiments, the MD 102, the e-tattoo 106, and/or the device 108 may be configured to sense intrathoracic impedance, from which various respiratory parameters may be derived, including, for example, respiratory tidal volume and minute ventilation. Sensors and associated circuitry may be incorporated in connection with the MD 102, the e-tattoo 106, and/or the device 108 for detecting one or more body movement or body posture and/or position related signals. For example, accelerometers, gyroscopes, and/or GPS devices may be employed to detect patient activity, patient location, body orientation, and/or torso position.

Derived parameters may also be monitored using the MD 102, the e-tattoo 106, and/or the device 108. For example, a respiration sensor may rely on measurements taken by an implanted accelerometer that measures body activity levels, respiration sounds, chest movement with respiration, heart sounds, and/or the like. The respiration sensor may include one or more electrodes configured to sense a physiological electrical signal, from which a respiration signal may be extracted. Respiration signals may additionally, or alternatively, be extracted from heart sound signals, cardiac electrical signals (e.g., electrograms), and/or the like. The respiration sensor may be used to estimate respiration patterns based on the measured parameters.

As stated above, one or more of the sensors 112, 114, 116 may be configured to sense physiological information about the subject 104. The physiological information may include at least one of: a respiration sensor, a sound sensor, a heart rate sensor, an oxygen sensor, a muscle use sensor, an activity sensor, a posture sensor, an inflammation sensor, a chemical sensor, an exhaled breath sensor, a thoracic composition sensor, an altered consciousness sensor, a central cyanosis sensor, and a sleep quality sensor. In embodiments, the MD 102, the e-tattoo 106, and/or the device 108 may include sensing components such as, for example, one or more surface electrodes configured to obtain an electrocardiogram (ECG), one or more accelerometers configured to detect motion associated with the subject 104, one or more respiratory sensors configured to obtain respiration information associated with the subject 104, one or more environmental sensors configured to obtain information about the external environment (e.g., temperature, air quality, humidity, carbon monoxide level, oxygen level, barometric pressure, light intensity, sound, and/or the like) to which the subject 104 is exposed, and/or the like. In embodiments, the MD 102, the e-tattoo 106, and/or the device 108 may be configured to measure parameters relating to the human body, such as temperature (e.g., a thermometer), blood pressure (e.g., a sphygmomanometer), blood characteristics (e.g., glucose levels), body weight, physical strength, mental acuity, diet, heart characteristics, relative geographic position (e.g., a Global Positioning System (GPS)), and/or the like.

Respiration sensors can be used to determine tidal volume (VT), respiration rate, peak expiratory flow rate (PEFR), forced expiratory volume (FEV), and a composite respiration index that includes at least one of an inspiration/expiration ratio (IER), VT times respiration rate, and respiration rate divided by VT. Respiration sensors may include any number of different types of sensors, including thoracic impedance sensors, accelerometers, flow sensors, and electrocardiograms (ECG or EKG). For example, the respiration rate can be sensed by one or more of a thoracic impedance sensor, an accelerometer, and an ECG. Also, the PEFR and the FEV can be determined using a thoracic impedance sensor to measure VT, and the IER can be determined using a thoracic impedance to measure VT. Other parameters associated with a respiratory functional test can also be used in determining asthma status. These parameters include the VT, FEV, and PEFR parameters, minute volume (MV), vital capacity (VC), functional residual capacity (FRC), total lung capacity, forced vital capacity (FVC), and forced expiratory flow (FEF).

Sound sensors can include at least one of a lung sound sensor, a speech sensor, and a heart sound sensor, where the lung sound sensor can be configured to sense wheezing in the patient. In embodiments, sound sensors include one or more of an accelerometer, a hydrophone, and a microphone. For example, a speech sensor and a lung sound sensor for sensing wheezing can include one or more of an accelerometer and a microphone.

In embodiments, a heart rate sensor includes an ECG for measuring the heart rate, an oxygen sensor includes an optical oxygen saturation sensor, and a central cyanosis sensor includes an optical oxygen saturation sensor. Also, in embodiments, a muscle use sensor and an activity sensor include one or more of a cervical and thoracic impedance sensor and an electromyogram for measuring activity. In addition, a posture sensor and an altered consciousness sensor include an accelerometer for measuring posture and/or balance. The inflammation sensor includes a chemical sensor for detecting an inflammatory marker, such as nitric oxide, and the sleep quality sensor includes one or more of a thoracic impedance sensor, an accelerometer, and an ECG for measuring tidal volume, respiration rate activity, posture, and heart rate. In embodiments, a sleep monitoring sensor may include an accelerometer that is incorporated into the e-tattoo 106 that is positioned on the eyelid of the subject 104.

In embodiments, a chemical sensor includes one or more of an inflammatory marker, e.g., a C-reactive protein, a pharmaceutical agent, e.g., theophylline, beta blockers, and/or aspirin, a blood gas, e.g., oxygen and/or carbon dioxide, and blood cell count, e.g., an eosinophil count. In embodiments, for example, a breath sensor include a chemical sensor such as, for example, a nitric oxide test, where increased levels of exhaled nitric oxide indicate inflammation, which can, for example, indicate a worsening asthma status.

In embodiments, the system 100 may also include a device 118 that may not be positioned adjacent the body of a subject 104, disposed on the body of the subject 104, and/or implanted within the body of a subject 104. In embodiments, the device 118 may store data (e.g., medical data) and/or provide data to any of the devices 102, 106, 108 via a communication link 110D. The data provided by the device 118 to one or more of the devices 102, 106, 108 may facilitate one or more of the devices 102, 106, 108 functioning as described above and below. In embodiments, the communication link 110D may be the same or similar to any of the communication links 110A, 110B, 110C.

The illustrative system 100 shown in FIG. 1 is not intended to suggest any limitation as to the scope of use or functionality of embodiments of the present disclosure. The illustrative system 100 should not be interpreted as having any dependency or requirement related to any single component or combination of components illustrated therein. Additionally, various components depicted in FIG. 1 may be, in embodiments, integrated with various ones of the other components depicted therein (and/or components not illustrated), all of which are considered to be within the ambit of the subject matter disclosed herein.

Various components depicted in FIG. 1 may operate together to form the system 100, which may be, for example, a computerized patient management and monitoring system. In embodiments, the system 100 may be designed to assist in monitoring the subject's condition, managing the subject's therapy, and/or the like. An illustrative patient management and monitoring system is the LATITUDE® patient management system from Boston Scientific Corporation, Marlborough Mass. Illustrative aspects of a patient management and monitoring system are described in ADVANCED PATIENT MANAGEMENT SYSTEM INCLUDING INTERROGATOR/TRANSCEIVER UNIT, U.S. Pat. No. 6,978,182 to Mazar et al., the entirety of which is hereby incorporated by reference herein.

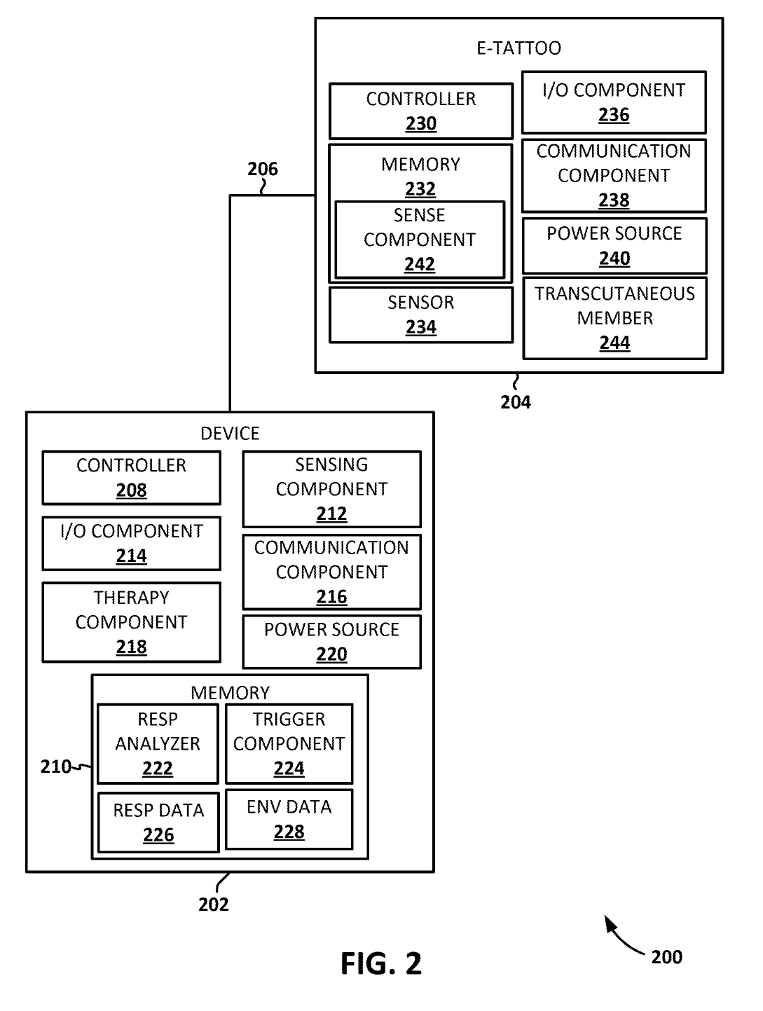

FIG. 2 is a block diagram depicting an illustrative operating environment 200, in accordance with embodiments of the subject matter disclosed herein. According to embodiments, the operating environment 200 may be, be similar to, include, be included in, or correspond to the system 100 depicted in FIG. 1. As shown in FIG. 2, the illustrative operating environment 200 includes a medical device (MD) 202 configured to communicate with an e-tattoo 204 via a communication link 206. In embodiments, the operating environment 200 may include the MD 202 without including an e-tattoo 204, include the e-tattoo 204 without including the MD 202, include another device (e.g., the device 108 depicted in FIG. 1) without included the MD 202, and/or include another device (e.g., the device 108 depicted in FIG. 1) without included the e-tattoo 204. According to embodiments, the operating environment 200 may include any number of other devices such as the device 108 depicted in FIG. 1 and/or any other types of devices, for example, additional medical devices, mobile devices, additional e-tattoos, and/or the like. According to embodiments, the MD 202 may be, be similar to, include, or be included in the MD 102 depicted in FIG. 1; the e-tattoo 204 may be, be similar to, include, or be included in the e-tattoo 106 depicted in FIG. 1; and, similarly, the communication link 206 may be, be similar to, include, or be included in the communication links 110A depicted in FIG. 1.

According to embodiments illustrated in FIG. 2, the MD 202 includes a controller 208, a memory 210, a sensing component 212, an input/output (I/O) component 214, a communication component 216, a therapy component 218, and/or a power source 220. The controller 208 may include, for example, a processing unit, a pulse generator, and/or the like. The controller 208 may be any arrangement of electronic circuits, electronic components, processors, program components and/or the like configured to store and/or execute programming instructions, to direct the operation of the other functional components of the MD 202, to perform respiratory functionality detection, ECG detection, EEG detection, EMG detection, arrhythmia detection and/or classification algorithms, to store physiologic data obtained by the sensing component 212, and/or the like, and may be implemented, for example, in the form of any combination of hardware, software, and/or firmware.

In embodiments, the controller 208 may be, include, or be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more central processing units (CPUs), software, hardware, firmware, or any combination of these and/or other components. According to embodiments, the controller 208 may include a processing unit configured to communicate with memory to execute computer-executable instructions stored in the memory. Although the controller 208 is referred to herein in the singular, the controller 208 may be implemented in multiple instances, distributed across multiple computing devices, instantiated within multiple virtual machines, and/or the like.

The controller 208 may also be configured to store information in the memory 210 and/or access information from the memory 210. The controller 208 may execute instructions and perform desired tasks as specified by computer-executable instructions stored in the memory 210. In embodiments, for example, the controller 208 may be configured to instantiate, by executing instructions stored in the memory 210, a respiratory (RESP) analyzer 222, and/or a trigger component 224. For example, in embodiments, the controller 208 may be configured to store and/or access respiration (RESP) data 226, environmental (ENV) data 228 (e.g., received from the e-tattoo 204), and/or the like.

In embodiments, the memory 210 includes computer-readable media in the form of volatile and/or nonvolatile memory and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In embodiments, the memory stores computer-executable instructions for causing the processor to implement aspects of embodiments of system components discussed herein and/or to perform aspects of embodiments of methods and procedures discussed herein.

The computer-executable instructions may include, for example, computer code, machine-useable instructions, and the like such as, for example, program components capable of being executed by one or more processors associated with the computing device. Program components may be programmed using any number of different programming environments, including various languages, development kits, frameworks, and/or the like. Some or all of the functionality contemplated herein may also, or alternatively, be implemented in hardware and/or firmware.

The MD 202 may sense physiological parameters using a sensing component 212 that may include, for example, one or more electrodes, one or more accelerometers, one or more thermometers, one or more chemical sensors, one or more pressure sensors, and/or the like. In embodiments, the sensing component 212 may include any number of electrical circuits, electronic components, processors, program components and/or the like. For example, in embodiments, the sensing component 212 may be configured to sense respiratory signals, and/or intrinsic cardiac electrical signals in a manner similar to known electrocardiogram (ECG) electrodes. In various embodiments, the sensing component 212 may be configured to sense other patient physiologic or environmental parameters in addition to, or alternative to, respiratory signals, and/or cardiac signals. In embodiments, the sensing component 212 may include temperature sensors (e.g., thermocouples or thermistors), barometers, acoustic sensors, pressure sensors, optical sensors, motion or impact sensors (e.g., accelerometers, inertial measuring units (IMUs)), strain sensors, Doppler systems, chemical sensors, ultrasound sensors, and/or the like, in any number of various types of configurations. The foregoing sensors allow the MD 202 to be capable of sensing and recording physiologic parameters such as, for example, patient movement, posture, respiratory cycles, heart sounds, and/or the like. The output from the sensing component 212 may be used in respiratory event detection and classification, therapy selection, trigger event detection, and/or the like.

The I/O component 214 may include and/or be coupled to a user interface configured to present information to a user or receive indication from a user. For example, the I/O component 214 may include and/or be coupled to a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like. In embodiments, the I/O component 214 may be used to present and/or provide an indication of any of the data sensed and/or produced by the MD 202

The communication component 216 may be configured to communicate (i.e., send and/or receive signals) with the e-tattoo and/or any other device (e.g., the device 108 depicted in FIG. 1). In embodiments, the communication component 216 may be configured to send a signal to the e-tattoo 204 in response to a trigger event being determined. Additionally or alternatively, the communication component 216 may be configured to receive signals from the e-tattoo 204 that are indicative of the one or more physiological and/or environmental parameters sensed by the e-tattoo 204. The communication component 216 may include, for example, circuits, program components, and one or more transmitters and/or receivers for communicating wirelessly with one or more other devices such as, for example, the e-tattoo 204. According to various embodiments, the communication component 216 may include one or more transmitters, receivers, transceivers, transducers, and/or the like, and may be configured to facilitate any number of different types of wireless communication such as, for example, radio-frequency (RF) communication, microwave communication, infrared communication, acoustic communication, inductive communication, conductive communication, and/or the like. The communication component 216 may include any combination of hardware, software, and/or firmware configured to facilitate establishing, maintaining, and using any number of communication links.

The therapy component 218 may be configured to delivery therapy in response to one or more sensed and/or derived signals, as discussed in more detail below. In embodiments, the therapy component 218 may include any number of different therapy components such as, for example, an inhaler component, a nebulizer component, a drug delivery component, defibrillation component, a neurostimulation component, a neuromodulation component, a temperature regulation component, and/or the like.

The power source 220 provides electrical power to the other operative components (e.g., the controller 208, the memory 210, the sensing component 212, the I/O component 214, the communication component 216, and the therapy component 218), and may be any type of power source suitable for providing the desired performance and/or longevity requirements of the MD 202. In various embodiments, the power source 220 may include one or more batteries, which may be rechargeable (e.g., using an external energy source). For example, in embodiments, e-tattoos may be used to charge implanted devices, transfer power from an external device to an implanted device (e.g., as a repeater, converter, amplifier, etc.), and/or the like. The power source 220 may include one or more capacitors, energy conversion mechanisms, and/or the like.

According to embodiments, sensed physiological parameters (sensed by the sensing component 212 and/or the e-tattoo 204) and/or ENV data 228 (obtained by the MD 202 and/or the e-tattoo 204) may be used to generate respiration data, activity data, and/or other data associated with a subject. For example, the MD 202 may include a respiration (RESP) analyzer 222 configured to analyze sensed signals to generate RESP data 226. Respiration parameters may be detected, for example, based on deflections and/or spectral analysis of a sensed signal such as, for example, an acceleration signal, a cardiac electrical signal, and/or the like. In embodiments, the MD 202 may be configured to develop, based on sensed data, a respiration profile associated with a subject, and may be configured to use the respiration profile to facilitate identification of respiratory events in additional respiration data. According to embodiments, sensed signals may be used to detect and/or characterize subject activity, which may facilitate sampling configuration and/or the like.

The RESP analyzer 222 may be configured to use various algorithms and mathematical modeling such as, for example, trend and statistical analysis, data mining, pattern recognition, cluster analysis, neural networks and/or fuzzy logic. The RESP analyzer 222 may perform deterministic and probabilistic calculations. Deterministic calculations include algorithms for which a clear correlation is known between the data analyzed and a given outcome.

As shown in FIG. 2, the MD 202 also may include a trigger component 224 configured to identify an occurrence of a trigger event. A trigger event is any designated event that, upon identification of its occurrence, results in some action. For example, the MD 202 may be configured (e.g., using the trigger component 224) to identify the occurrence of a trigger event, and obtain sensed data via the sensing component 212 and/or generate the RESP data 226 via the RESP analyzer 222 in response to identifying the occurrence of the trigger event. In embodiments, the MD 202 may be configured to identify the occurrence of a trigger event by receiving an indication of a user input; determining that a state of a parameter (e.g., a physiological parameter, an environmental parameter, and/or a device parameter) satisfies one or more trigger criteria; and/or the like.

According to embodiments, the trigger event may include an occurrence of a specified characteristic occurring physiological parameter. For example, in embodiments, the RESP analyzer 222 may be configured to receive sensing data (e.g., from the sensing component 212, the e-tattoo 204, etc.), generate RESP data 226, and/or otherwise perform some designated task in response to the trigger component 224 identifying the occurrence of a trigger event. For example, the sensing component 212 may sense abnormal breathing by a subject, which may be a trigger event for the trigger component 224. In response, the trigger component 224 may instruct the RESP analyzer 222 to start analyzing the sensed data to produce RESP data 226. Similarly, in embodiments, the RESP analyzer 222 may be configured to receive respiratory parameters from a sensing component 212, obtain ENV data 228, and/or otherwise perform some designated task in response to identifying the occurrence of a trigger event. The RESP data 226 produced may include the parameters of the abnormal breathing (e.g., rate, depth, rhythm). Additionally or alternatively, the RESP data 226 produced in response to a trigger event may be used to determine therapy to be delivered to a subject by the therapy component 218.

Additionally or alternatively, the trigger component 224 may instruct the MD 202 to send a signal to the e-tattoo 204 in response to determining a trigger event. In response to receiving the signal from the MD 202, the e-tattoo 204 may start sensing and storing data, as described in more detail below.

According to embodiments, the trigger component 224 may be configured to implement any number of different adjudication algorithms to detect a trigger event. The trigger component 224 may detect a trigger event based on information received from any number of other components, devices, and/or the like. For example, the trigger component 224 may obtain physiological and/or environmental parameter information from the e-tattoo 204 and may use that parameter information to detect a trigger event. Trigger events may be user defined, system defined, statically defined, dynamically defined, and/or the like. The trigger component 224 may reference trigger criteria stored in memory 210 to determine whether a trigger event has occurred. The trigger criteria may be established by a clinician, a patient, an algorithm, and/or the like.

In embodiments, to detect a trigger event based on a first trigger event, the trigger component 224 may also be configured to obtain information associated with a second trigger event. A set of trigger criteria also may be dynamically adapted over time, using a machine-learning process. That is, for example, as a subject ages, adopts changes to daily routines (e.g., diet, exercise, sleep habits, etc.), and/or the like, the trigger component 224 may dynamically adapt trigger criteria so that, for example, a smaller level of activity may be detected as a trigger event when the patient is older than when the patient was younger. Additionally, machine-learning techniques may be employed to adapt trigger criteria to more rapidly-changing scenarios such as, for example, the impact of adjusting to a new medication, the impact of a temporary adjustment in sleep schedule, the impact of the air quality in a particular location (e.g., outside vs. inside, downtown vs. at home, one city vs. another, etc.), the impact of an allergic reaction to an environmental stimulus, the impact of a psychological response to an increase or decrease in an amount of sunlight over the course of one or more days, the impact of a rapid change in barometric pressure, and/or the like. According to embodiments, adapting a set of trigger criteria may include adjusting one or more thresholds, adjusting one or more value ranges, adding or subtracting types of information to be considered (e.g., requiring additional, or fewer, inputs to an adjudication algorithm), adjusting weight applied to one or more inputs, adjusting error terms, adjusting boundary conditions, and/or the like.

Using specified criteria, the trigger component 224 can be configured to differentiate between trigger events and other events that may be accidental, or otherwise natural, occurrences such as, for example, heart beats, vibrations and other measurable changes caused by the patient engaging in activity (e.g., impacts between the patient's feet and the ground), signals caused by sound waves impinging on the surface of the patient's body (e.g., at concerts, in theaters, etc.), and/or the like. In embodiments, the trigger component 224 may be further configured to cause an I/O component 214 (and/or an I/O component of the e-tattoo 204) to present an indication that a trigger event has occurred.

As shown in FIG. 2, the e-tattoo 204 includes a controller 230, a memory 232, a sensor 234, an I/O component 236, a communication component 238, and a power source 240. The controller 230 may include, for example, a processing unit, a pulse generator, and/or the like. The controller 230 may be any arrangement of electronic circuits, electronic components, processors, program components and/or the like configured to store and/or execute programming instructions, to direct the operation of the other functional components of the e-tattoo 204, to perform respiratory functionality detection, ECG detection, EEG detection, EMG detection, arrhythmia detection and/or classification algorithms, to store physiologic data obtained by the sensor 234, and/or the like, and may be implemented, for example, in the form of any combination of hardware, software, and/or firmware.

In embodiments, the controller 230 may be, include, or be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more central processing units (CPUs), software, hardware, firmware, or any combination of these and/or other components. According to embodiments, the controller 230 may include a processing unit configured to communicate with memory to execute computer-executable instructions stored in the memory. Although the controller 230 is referred to herein in the singular, the controller 230 may be implemented in multiple instances, distributed across multiple computing devices, instantiated within multiple virtual machines, and/or the like.

The controller 230 may also be configured to store information in the memory 232 and/or access information from the memory 232. The controller 230 may execute instructions and perform desired tasks as specified by computer-executable instructions stored in the memory 232. In embodiments, for example, the controller 230 may be configured to instantiate, by executing instructions stored in the memory 232, a sense component 242 and/or the like. Additionally or alternatively, the controller 230 may store any data sensed by the sensor 234 to the memory 232. Additionally or alternatively, if the sensed data is transferred from the e-tattoo 204 to another device, the controller 230 may be configured to erase the sensed data from the e-tattoo 204 to free-up storage space on the memory 232.

In embodiments, the memory 232 includes computer-readable media in the form of volatile and/or nonvolatile memory and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In embodiments, the memory stores computer-executable instructions for causing the processor to implement aspects of embodiments of system components discussed herein and/or to perform aspects of embodiments of methods and procedures discussed herein.

The computer-executable instructions may include, for example, computer code, machine-useable instructions, and the like such as, for example, program components capable of being executed by one or more processors associated with the computing device. Program components may be programmed using any number of different programming environments, including various languages, development kits, frameworks, and/or the like. Some or all of the functionality contemplated herein may also, or alternatively, be implemented in hardware and/or firmware.

The e-tattoo 204 may sense various physiological and/or environmental parameters using a sensor 234. The environmental parameters may include particulates, ultraviolet light, volatile organic compounds, and/or the like in the environment. The physiological parameters may include respiratory parameters (e.g., rate, depth, rhythm), motion parameters, (e.g., walking, running, falling, gait, gait rhythm), facial expressions, swelling, heart sounds, sweat, sweat composition (e.g., ammonia, pH, potassium, sodium, chloride), exhaled air composition, Electrocardiography (ECG) parameters, electroencephalogram (EEG) parameters, Electromyography (EMG) parameters, and/or the like. To sense the one or more environmental parameters and/or physiological parameters, the sensor 234 may include temperature sensors (e.g., thermocouples or thermistors), barometers, acoustic sensors, pressure sensors, optical sensors, motion or impact sensors (e.g., accelerometers, gyroscopes, inertial measuring units (IMUs)), strain sensors, Doppler systems, chemical sensors, ultrasound sensors, and/or the like, in any number of various types of configurations.

In embodiments, the e-tattoo 106 may have a transcutaneous member 244 piercing the skin of subject (e.g., subject 104). The transcutaneous member 244 may contain one or more sensors measuring parameters within a subject (i.e. a blood parameter, an interstitial fluid parameter, an electrical parameter). The transcutaneous member 244 may contain one or more components (e.g. an electrode, a catheter) for delivering one or more therapies (e.g. a neurostimulation therapy, a drug therapy). In an embodiment, the transcutaneous member 244 may measure glucose and deliver insulin.

The I/O component 236 may include and/or be coupled to a user interface configured to present information to a user or receive indication from a user. For example, the I/O component 236 may include and/or be coupled to a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, a volatile compound release depot, and/or the like. In embodiments, the I/O component 236 may be used to present and/or provide an indication of any of the data sensed and/or produced by the device 202 and/or e-tattoo 204. According to embodiments, for example, the I/O component 236 may include one or more visual indicators (e.g., single-color LED lights, multi-color LED lights, a flexible digital display device, and/or the like) configured to provide information to a user (e.g., by illuminating, flashing, displaying data, etc.). Additionally or alternatively, the I/O component 236 may be used to control therapy provided by the device 202 and/or e-tattoo 204.

The communication component 238 may be configured to communicate (i.e., send and/or receive signals) with the MD 202 and/or any other device (e.g., the device 108 depicted in FIG. 1). Additionally or alternatively, any data sensed by the sensor 234 may be transmitted to the MD 202 for processing and/or storage.

In embodiments, the communication component 238 may include, for example, circuits, program components, and one or more transmitters and/or receivers for communicating wirelessly with one or more other devices such as, for example, the MD 202. According to various embodiments, the communication component 238 may include one or more transmitters, receivers, transceivers, transducers, and/or the like, and may be configured to facilitate any number of different types of wireless communication such as, for example, radio-frequency (RF) communication, microwave communication, infrared or visual spectrum communication, acoustic communication, inductive communication, conductive communication, and/or the like. The communication component 238 may include any combination of hardware, software, and/or firmware configured to facilitate establishing, maintaining, and using any number of communication links.

The power source 240 provides electrical power to the other operative components (e.g., the controller 230, the memory 232, the sense component 242, the I/O component 236, and the communication component 238), and may be any type of power source suitable for providing the desired performance and/or longevity requirements of the e-tattoo 204. In various embodiments, the power source 240 may include one or more batteries, which may be rechargeable (e.g., using an external energy source). The power source 240 may include one or more capacitors, energy conversion mechanisms, and/or the like. Additionally or alternatively, the power source 240 may harvest energy from a subject (e.g., the subject 104) (e.g. motion, heat, biochemical) and/or from the environment (e.g. electromagnetic). Additionally or alternatively, the power source 240 may harvest energy from an energy source connected to the body, for example, a shoe may receive energy from impact and send the received energy to a power source 240 of the e-tattoo 204.

In embodiments, the power source 240 may transfer power to the power source 220 using a wireless or non-wireless connection (e.g., via conduction, induction, radio-frequency, etc.). Because the MD 202 may be implanted within a subject and it may be hard to remove the MD 202 from the subject, the longevity of the MD 202 may be increased via power transfer from the e-tattoo 204 to the MD 202. Additionally or alternatively, the power source 220 may transfer power to the power source 240 in order to increase the longevity of the e-tattoo 204.

According to embodiments, the sense component 242 may be configured to provide a signal to the sensor 234 to begin sensing one or more physiological and/or environmental parameters. In embodiments, the signal may be provided by the sense component 242 to the sensor 234 before the therapy component 218 provides and/or modifies a therapy to a subject in order to establish one or more baseline measurement of one or more physiological and/or environmental parameters. Additionally or alternatively, the signal may be provided by the sense component 242 to the sensor 234 after the therapy component 218 provides and/or modifies a therapy to a subject in order to establish non-baseline measurements of the one or more physiological and/or environmental parameters. By sensing baseline and non-baseline measurements of one or more physiological and/or environmental parameters, a comparison of the baseline and non-baseline measurements may be performed in order to determine the effectiveness of the therapy administered by the therapy component 218 may be determined. In embodiments, the comparison may be performed by the sense component 242. Additionally or alternatively, the sense component 242 may be configured to initiate transmitting the baseline and non-baseline measurements back to the MD 202 via the communication component 238, so that the MD 202 (e.g., the RESP analyzer 222) can perform the comparison. Additionally or alternatively, a therapy provided by therapy component 218 of the MD 202 may be altered based on the comparison.

Additionally or alternatively, the sense component 242 may provide a signal to the sensor 234 in response to the communication component 238 receiving a signal from the MD 202. In embodiments, the signal may be indicative of one or more trigger events occurring. In embodiments, the e-tattoo 204 may also be positioned in a more advantageous position to sense one or more physiological and/or environmental parameters than the MD 202. Additionally or alternatively, by waiting to begin sensing until a trigger event has been determined, power of the power source 240 may be better managed.

The illustrative operating environment shown in FIG. 2 is not intended to suggest any limitation as to the scope of use or functionality of embodiments of the present disclosure. The illustrative operating environment 200 also should not be interpreted as having any dependency or requirement related to any single component or combination of components illustrated therein. Additionally, various components depicted in FIG. 2 may be, in embodiments, integrated with various ones of the other components depicted therein (and/or components not illustrated), all of which are considered to be within the ambit of the present disclosure.

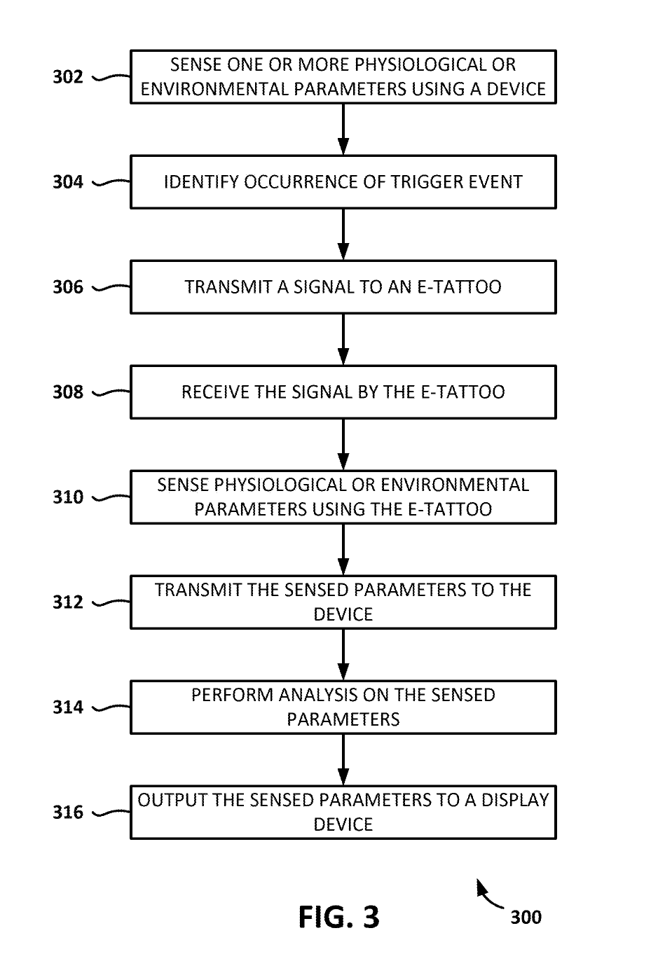

FIG. 3 is a flow diagram depicting an illustrative method 300 for monitoring a subject (e.g., the subject 104) and/or a subject's environment, in accordance with embodiments disclosed herein. In embodiments method 300 may monitor, for example, a chronic medical condition (e.g. heart failure, COPD, asthma, diabetes, cancer), an acute medical condition (e.g. infection, surgery recovery, stroke, myocardial infarction), a fitness level and/or a risk of acquiring a medical condition (e.g. cancer, cardiovascular disease) of a subject. According to embodiments, the method 300 may be performed by any number of different aspects of components of the system 100 depicted in FIG. 1 and/or the operating environment 200 depicted in FIG. 2. For example, in embodiments, the illustrative method 300 may be performed by an MD (e.g., the MD 102 depicted in FIG. 1 and/or the MD depicted in FIG. 2), an e-tattoo (e.g., the e-tattoo 106 depicted in FIG. 1 and/or the e-tattoo 204 depicted in FIG. 2), and/or a device (the device 108 depicted in FIG. 1), as described herein.

Embodiments of the method 300 may include sensing one or more physiological or environmental parameters using a device (block 302). The one or more parameters may include, but are not limited to: particulates, ultraviolet light, volatile organic compounds, respiratory parameters (e.g., rate, depth, rhythm), motion parameters, (e.g., walking, running, falling, gait, gait rhythm), facial expressions, swelling, heart sounds, sweat, sweat composition (e.g., ammonia, pH, potassium, sodium, chloride), exhaled air composition, Electrocardiography (ECG) parameters, electroencephalogram (EEG) parameters, and/or Electromyography (EMG) parameters.

According to embodiments, the method 300 may include identifying a trigger event included in the sensed parameters (block 304). An example of a trigger event may be abnormal breathing. In response to determining a trigger event, a signal may be transmitted from the device to an e-tattoo (block 306), which the e-tattoo may receive (block 308).

Upon receiving a signal that a trigger event occurred, the e-tattoo may be configured to sense one or more physiological or environmental parameters (block 310). The parameters sensed by the e-tattoo may be the same or similar to the parameters sensed by the device. Alternatively, the parameters sensed by the e-tattoo may be different than the parameters sensed by the device. For example, in embodiments, the device may be configured to sense physiological parameters and the e-tattoo may be configured to sense environmental parameters. In embodiments, the method 300 may also include the e-tattoo transmitting the sensed parameters to the device and/or another device (block 312). In embodiments, the device may perform additional analysis on the sensed parameters (block 314) and/or the sensed parameters may be output to a display device (block 316).

The illustrative method 300 shown in FIG. 3 is not intended to suggest any limitation as to the scope of use or functionality of embodiments of the present disclosure. The illustrative method 300 should not be interpreted as having any dependency or requirement related to any single block or combination of blocks illustrated therein. Additionally, various blocks depicted in FIG. 3 may be, in embodiments, integrated with various ones of the other blocks depicted therein (and/or components not illustrated), all of which are considered to be within the ambit of the subject matter disclosed herein.

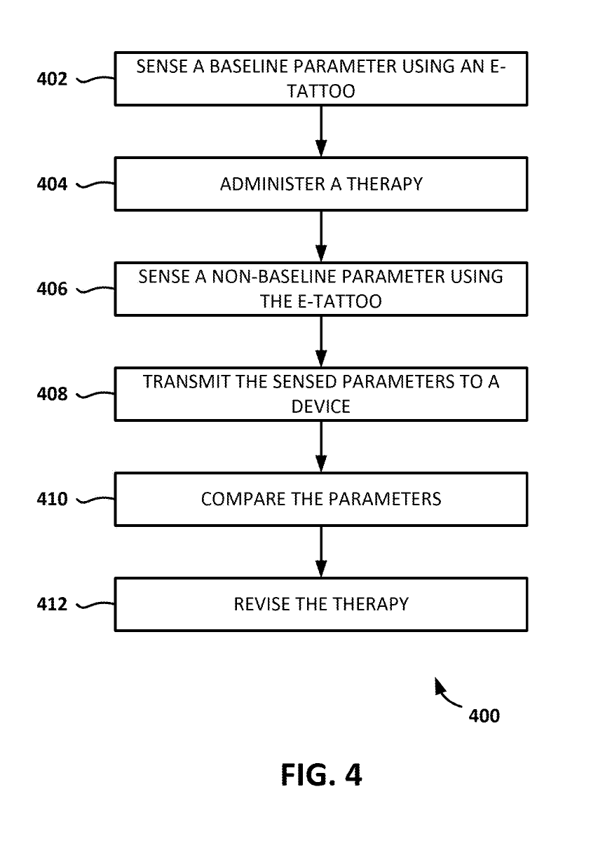

FIG. 4 is a flow diagram depicting another illustrative method 400 for monitoring a subject (e.g., the subject 104) and/or a subject's environment, in accordance with embodiments disclosed herein. According to embodiments, the method 400 may be performed by any number of different aspects of components of the system 100 depicted in FIG. 1 and/or the operating environment 200 depicted in FIG. 2. For example, in embodiments, the illustrative method 400 may be performed by an MD (e.g., the MD 102 depicted in FIG. 1 and/or the MD depicted in FIG. 2), an e-tattoo (e.g., the e-tattoo 106 depicted in FIG. 1 and/or the e-tattoo 204 depicted in FIG. 2), and/or a device (e.g., the device 108 depicted in FIG. 1), as described herein. Additionally or alternatively, one or more aspects of method 400 may be combined and/or interchanged with one or more aspects of method 300.

Embodiments of the method 400 may include sensing one or more baseline measurements of one or more parameters using an e-tattoo (block 402). The parameters may include, but are not limited to: particulates, ultraviolet light, volatile organic compounds, respiratory parameters (e.g., rate, depth, rhythm), motion parameters, (e.g., walking, running, falling, gait, gait rhythm), facial expressions, swelling, heart sounds, sweat, sweat composition (e.g., ammonia, pH, potassium, sodium, chloride), exhaled air composition, Electrocardiography (ECG) parameters, electroencephalogram (EEG) parameters, and/or Electromyography (EMG) parameters.

In embodiments, the method 400 may include administering a therapy (block 404). In embodiments, the therapy may be administered using a device. Additionally or alternatively, a MD may be implanted into a subject after one or more baseline measurements of one or more parameters are sensed. The method 400 may further include sensing one or more non-baseline measurements of the one or more parameters using the e-tattoo (block 406). The one or more parameters that are sensed after a therapy may be the same parameters that are sensed before the therapy, but may be sensed again to determine the effectiveness of the therapy. The method 400 may include transmitting the sensed parameters to a device (block 408). The device may then compare the sensed parameters to determine the effectiveness of the therapy (block 410). In embodiments, the method 400 may include modifying the therapy based on the comparison (block 412).

The illustrative method 400 shown in FIG. 4 is not intended to suggest any limitation as to the scope of use or functionality of embodiments of the present disclosure. The illustrative method 400 should not be interpreted as having any dependency or requirement related to any single block or combination of blocks illustrated therein. Additionally, various blocks depicted in FIG. 4 may be, in embodiments, integrated with various ones of the other blocks depicted therein (and/or components not illustrated), all of which are considered to be within the ambit of the subject matter disclosed herein.

Various modifications and additions can be made to the exemplary embodiments discussed without departing from the scope of the disclosed subject matter. For example, while the embodiments described above refer to particular features, the scope of this disclosure also includes embodiments having different combinations of features and embodiments that do not include all of the described features. Accordingly, the scope of the subject matter disclosed herein is intended to embrace all such alternatives, modifications, and variations as fall within the scope of the claims, together with all equivalents thereof.

We claim:

1. A system for monitoring one or more respiratory conditions, the system comprising:
a medical device configured to:
monitor at least one respiratory parameter of a subject; and
transmit a signal in response to monitoring a trigger event in the at least one respiratory parameter; and
an e-tattoo configured to attach to the subject's skin, the e-tattoo configured to:
receive the signal from the medical device;
sense at least one environmental parameter in response to receiving the signal; and
transmit the at least one environmental parameter.

2. The system of claim 1, wherein the medical device is configured to receive the at least one transmitted environmental parameter.

3. The system of claim 2, wherein the medical device is configured to modify a therapy in response to receiving the at least one transmitted environmental parameter.

4. The system of claim 2, wherein the medical device is further configured to:
determine the at least one environmental parameter is an adverse environmental parameter; and
provide, to a subject, an indication the at least one environmental parameter is an adverse environmental parameter.

5. The system of claim 1, wherein the at least one environmental parameter is at least one of: a particulate and a volatile organic compound.

6. The system of claim 1, wherein the trigger event is at least one of: a change in a respiratory sound and a change in respiratory rate.

7. The system of claim 1, wherein the e-tattoo is further configured to:
sense a baseline measurement of a respiratory parameter, wherein the baseline measurement is sensed prior to the medical device modifying a therapy;
sense a non-baseline measurement of the respiratory parameter, wherein the non-baseline measurement is sensed after the medical device modifies a therapy; and
transmit the baseline measurement and the non-baseline measurement to a device.

8. The system of claim 7, wherein the device is configured to:
compare the baseline measurement and the non-baseline measurement; and
modify the therapy for the subject in response to the comparison.

9. The system of claim 7, further comprising a display device communicatively coupled to at least one of: the medical device and the e-tattoo, the display device configured to present a representation of at least one of: the at least one environmental parameter, the baseline measurement, and the non-baseline measurement.

10. A method for monitoring one or more respiratory conditions, the method comprising:
receiving, by an e-tattoo configured to attach to a subject's skin, a signal transmitted from a medical device, wherein the signal is transmitted in response to detecting a trigger event in at least one respiratory parameter of a subject;
sensing at least one environmental parameter in response to receiving the signal; and
transmitting the at least one environmental parameter.

11. The method of claim 10, further comprising:
sensing a baseline measurement of a respiratory parameter, wherein the baseline measurement is sensed prior to the medical device modifying a therapy;
sensing a non-baseline measurement of the respiratory parameter, wherein the non-baseline measurement is sensed after the medical device delivers the therapy; and
transmitting the baseline measurement and the non-baseline measurement of a respiratory parameter to a device.

12. The method of claim 11, further comprising:
comparing the baseline measurement and the non-baseline measurement; and
modifying the therapy for the subject in response to the comparison.

13. The method of claim 11, further comprising displaying a representation of at least one of: the at least one environmental parameter, the baseline measurement, and the non-baseline measurement.

14. The method of claim 10, wherein the trigger event is at least one of: a change in a respiratory sound and a change in respiratory rate.

15. The method of claim 10, wherein the at least one environmental parameter is at least one of: a particulate and a volatile organic compound.

16. An e-tattoo comprising:
an adhering mechanism for attaching the e-tattoo to a subject's skin; and
a processing device configured to:
receive an signal from a medical device;
sense at least one environmental parameter in response to receiving the signal; and
transmit the at least one environmental parameter.

17. The e-tattoo of claim 16, wherein the processing device is further configured to:
sense a baseline measurement of a respiratory parameter, wherein the baseline measurement is sensed prior to the medical device modifying a therapy;
sense a non-baseline measurement of the respiratory parameter, wherein the non-baseline measurement is sensed after the medical device modifies a therapy; and
transmit the baseline measurement and the non-baseline measurement to a device.

18. The e-tattoo of claim 16, wherein the processing device is further configured to:
compare the baseline measurement and the non-baseline measurement and transmit the comparison to the device.

19. The e-tattoo of claim 16, wherein the processing device is further configured to:
determine the at least one environmental parameter is an adverse environmental parameter; and
transmit a signal to the device indicating the determined adverse environmental parameter.

20. The e-tattoo of claim 16, wherein the at least one environmental parameter is at least one of: a particulate and a volatile organic compound.
